(19) United States
(12) Patent Application Publication
(10) Pub. No.: US 2022/0249325 A1
(43) Pub. Date: Aug. 11, 2022

(54) Medicament Container

(71) Applicant: Sanofi, Paris (FR)

(72) Inventors: Silke Dittombee, Frankfurt am Main (DE); Sebastian Blum, Frankfurt am Main (DE); Katharina Lenk, Frankfurt am Main (DE); Stefanie Ulrike Becker, Frankfurt am Main (DE)

(21) Appl. No.: 17/616,398

(22) PCT Filed: Jun. 4, 2020
(86) PCT No.: PCT/EP2020/065403

§ 371 Date: Dec. 3, 2021

(30) Foreign Application Priority Data
Jun. 5, 2019 (EP) 19305724.7

(51) Int. Cl. A61J 1/06; A61M 5/24; A61M 5/31

(52) U.S. Cl. CPC A61J 1/062; A61M 5/2466; A61M 5/3134; A61M 5/2422; A61M 2005/2403; A61M 2005/3103; A61M 2005/3118; A61M 2207/10; A61M 2005/2437

(57) ABSTRACT

The present disclosure relates to a medicament container for accommodating a liquid medicament. The medicament container includes an elongated container barrel defining a longitudinal direction and having a proximal end and a distal end. The proximal end is opposite the distal end. The container barrel includes a proximal container cross section at the proximal end. The elongated container barrel includes a distal container cross section at the distal end and the proximal container cross section is substantially identical to the distal container cross section. The medicament container further includes a seal arranged at the distal end and extending across the distal container cross section, and a retainer engaged with the seal and engaged with the elongated container barrel to fix the seal to the distal end.

### CROSS REFERENCE TO RELATED APPLICATIONS

The present application is the national stage entry of International Patent Application No. PCT/EP2020/065403, filed on Jun. 4, 2020, and claims priority to Application No. EP 19305724.7, filed on Jun. 5, 2019, the disclosures of which are incorporated herein by reference.

### TECHNICAL FIELD

The present disclosure relates to the field medicament containers, such as cartridges, carpules, vials or prefilled syringes. In particular, the disclosure relates to a primary packaging configured to accommodate a liquid medicament. The primary packaging, such as a cartridge, a vial, a carpule or a pre-filled syringe is typically filled with a liquid medicament, e.g. with an injectable liquid medicament.

### BACKGROUND

Drug delivery devices for setting and dispensing a single dose or multiple doses of a liquid medicament are as such well-known in the art. Generally, such devices have substantially a similar purpose as that of an ordinary syringe.

Drug delivery devices, such as pen-type injectors have to meet a number of user-specific requirements. For instance, with patient's suffering chronic diseases, such as diabetes, the patient may be physically infirm and may also have impaired vision. Suitable drug delivery devices especially intended for home medication need to be robust in construction and easy to use. Furthermore, manipulation and general handling of the device and its components should be intelligible and easy understandable. Injection devices can provide setting and subsequent dispensing of a dose of a medicament of variable size. It is advantageous for, a dose setting as well as a dose dispensing procedure to be easy to operate and to be unambiguous.

Typically, such devices comprise a housing or a holder, e.g. a cartridge holder configured to receive a medicament container, e.g. in form of a cartridge at least partially filled with the medicament to be expelled. The device further comprises a drive mechanism, usually having a displaceable plunger or piston rod to operably engage with a bung or piston of the medicament container or cartridge. By means of the drive mechanism and the piston rod, the bung or piston of the cartridge is displaceable in a distal direction or dispensing direction and may be therefore configured to expel a predefined amount of the medicament via a piercing assembly, e.g. in form of an injection needle, connected to or releasably connectable with an outlet end of the medicament container.

With reusable drug delivery devices an empty cartridge is replaceable by a filled one. In contrast to that, drug delivery devices of disposable type are to be entirely discarded when the medicament in the cartridge has been dispensed or used-up.

In general, medicament containers, such as cartridges, carpules or pre-filled syringes typically comprise a barrel of substantially tubular shape having an outlet at a distal end and having a bung or a piston moveably arranged inside the barrel. The bung or piston seals the barrel towards a proximal end opposite to the distal outlet end. By displacing the bung towards the distal end a predefined amount of the medicament, i.e. a dose of the medicament, can be expelled through the outlet of the cartridge.

A conventional cartridge 40 as known in the prior art is schematically illustrated in FIG. 3. The cartridge comprises a barrel 41 with a tubular body. Towards a proximal end the barrel 41 is sealed by a piston 42 displaceable inside the barrel 41 along a longitudinal direction in order to expel the liquid medicament via an outlet at the opposite distal end of the barrel 41. The distal end of the barrel 41 opposite to the piston 42 is sealed by a pierceable seal 45. The pierceable seal 45 is fixed to a diameter reduced head portion 44 of the barrel 41 by a cap 46. The outlet end of the cartridge 40 typically comprises a radially stepped down head portion 44 extending via a neck portion 43 and/or a shoulder portion into the tubular shaped main part or body of the barrel 41.

Typically, such cartridges 40 are made from a vitreous material, e.g. glass. The production process of such vitreous barrels 41 is rather elaborate. The geometry of the barrel 41 in the region of the diameter reduced neck portion 43 and the diameter reduced head portion 44 as well as in the shoulder region is inevitably subject to comparatively large geometric tolerances.

Moreover, with the stepped down neck portion, the shoulder portion and the stepped down head portion a complete filling of the available interior volume of the cartridge 40 with a liquid medicament might be quite difficult. After the cartridge has been filled, the seal 45 and the cap 46, e.g. implemented as a crimp cap have to be manually placed and fastened to the head portion 44. The handling of filled and non-sealed cartridges is quite elaborate. Moreover, a filling of the interior volume of the cartridge 40 without any gaseous components, hence an airless filling is difficult to obtain.

With cartridges 40 featuring a radially stepped down shoulder portion or neck portion 43 a complete emptying of the cartridge 40 by urging the piston 42 towards the distal seal 45 is also quite difficult; yet impossible. Due to the radially stepped down neck portion 43 and the stepped down head portion 44 the piston 42 can never get in direct contact with a proximal face of the seal 45. Insofar there always remains a residual amount of the medicament that is left in the cartridge.

### SUMMARY

An aspect of the present disclosure provides an improved medicament container that overcomes the above described disadvantages. One implementation provides a medicament container that enables a rather easy, intuitive and fast assembly. The medicament container can include a rather robust medicament container equally suitable for long term storage of a liquid medicament as well as for administering the liquid medicament. The medicament container can enable a complete emptying of the container with a minimum of a residual amount of the medicament left in the medicament container after use thereof. Moreover, the medicament container can provide and support a rather precise manufacturing and assembly.

The medicament container can be characterized by improved and/or reduced geometric tolerances. The medicament container can also enable an easy and straightforward filling with the liquid medicament.

In one aspect there is provided a medicament container for accommodating a liquid medicament. The medicament container comprises an elongated container barrel defining a longitudinal direction. The elongated container barrel comprises a proximal end and a distal end. The proximal end is opposite to the distal end. The proximal end and the distal end are separated along the longitudinal direction of the container barrel. The container barrel comprises a proximal container cross-section at the proximal end. The elongated container barrel further comprises a distal container cross-section at the distal end. The proximal container cross-section is substantially identical to the distal container cross-section.

The medicament container further comprises a seal arranged at the distal end and extending across the distal container cross-section. Typically, the seal serves to seal the distal container cross-section in a fluid and/or gas-tight manner. The medicament container further comprises a retainer. The retainer is engaged with the seal and is also engaged with the elongated container barrel to fix the seal to the distal end. Typically, the seal is fixed to the distal end and/or across the distal container cross-section by the retainer.

With some examples the elongated container barrel is void of a stepped down neck portion, a stepped down head portion and/or of a stepped down shoulder portion. In particular, the elongated container barrel is void of any radially stepped down portions at a distal end, wherein the distal end is the outlet end of the medicament container. The container barrel may be of rigid structure. Hence, it may comprise a substantially inelastic or non-deformable material.

With some examples the elongated container barrel is of tubular or cylindrical shape. The proximal end and the distal end of the elongated container barrel typically comprise a substantially identical cross-sections, in particular substantially identical inner cross-sections. Moreover, a diameter of the proximal end of the elongated container barrel is substantially identical or absolutely identical with the diameter at the distal end of the elongated container barrel. In this way a piston, e.g. slidably arranged inside the elongated container barrel can be displaced along the entire elongation or longitudinal direction of the elongated container barrel until it directly engages with the seal at the distal end of the elongated container barrel. This allows and supports a complete residualless emptying of the medicament container for and during administering of a liquid medicament.

Typically and with some examples the elongated container barrel comprises a sleeve shape with a constant inner and/or outer diameter and/or with a constant inner and/or outer cross-section along its longitudinal direction. From the proximal end towards the distal end neither the inner cross-section nor the outer cross-section may be subject to any modifications or variations. The elongated container barrel typically comprises a sidewall connecting the proximal end and the distal end. The sidewall comprises a constant thickness from the proximal end towards the distal end and vice versa.

With the tubular-shaped and sleeve-like elongated container barrel geometric tolerances of the elongated container barrel and the medicament container can be reduced. Moreover, the manufacturing of the sleeve-shaped shoulderless, neckless and/or headless elongated container barrel is rather simple and straight forward. Manufacturing costs and manufacturing expenditure can be therefore reduced.

Moreover, the tubular shape of the elongated container barrel also provides an easier handling of the elongated container barrel and an easier mutual assembly of the elongated container barrel, the seal and the retainer in the course of manufacturing of the medicament container. In addition, a filling process for filling the interior volume of the elongated container can be simplified and improved. Since the elongated container barrel is shoulderless, neckless and/or headless also its longitudinal position inside an injection device can be defined in a more accurate way. Geometric tolerances or assembly tolerances regarding the positioning or arrangement of the medicament container inside a drug delivery device or inside an injection device with regard to the longitudinal direction can be reduced. In addition, the longitudinal position of a piston located inside the elongated container with regard to a piston rod or plunger rod of a drive mechanism of a drug delivery device or of an injection device can be provided with higher precision when the medicament container is arranged or assembled in the drug delivery device.

According to a further example the retainer comprises a proximal fastener engaged with the proximal end of the elongated container barrel. The proximal fastener of the retainer enables a longitudinal squeezing or clamping of the elongated container barrel between the seal and the proximal fastener of the retainer. The proximal fastener may be implemented as a positive fit configured to positively engage with the proximal end of the elongated container barrel.

With other examples the proximal fastener provides at least one of a force fitted fastening and a friction fitted fastening. The retainer may extend from the distal end towards the proximal end of the elongated container barrel. Typically, the retainer extends along the outside of the container barrel. The retainer is typically made of a material being resistant to traction. Moreover, the material of the retainer is tension stable. A tensionally stable retainer serves to provide a longitudinal clamping of the seal to or at the distal end of the elongated container.

When implemented as a form fitted fastener the retainer may form or may provide a clip or snap joint with the proximal end of the elongated container barrel. The proximal end of the elongated container barrel may therefore be connected and fixed or fastened with the elongated retainer or elongated retainer barrel by way of a clip or snap feature. This allows and provides a rather simple, intuitive and straight forward mutual assembly of the elongated container barrel, the seal and the retainer.

In a further example the proximal fastener comprises a distally facing abutment face configured to abut in longitudinal direction with a proximal end face of the container barrel. The distally facing abutment face belongs to the proximal fastener of the retainer. When appropriately assembled, the distally facing abutment face is configured to engage with the proximal end face of the container barrel thereby impeding and/or preventing a proximally directed movement or displacement of the container barrel relative to the retainer. In this way the distally facing abutment face of the proximal fastener provides a proximal end stop for the proximal end face and hence of the proximal end of the container barrel. Once the distally facing abutment face is engaged or is in abutment with the proximal end face of the container barrel the container barrel can no longer move in proximal direction beyond the proximal fastener.

According to another example the retainer comprises a distal fastener engaged with at least one of the distal end of the elongated container barrel and the seal. The distal fastener may engage with at least one of the distal end of the elongated container barrel and the seal in a form fitted, in a force fitted and/or in a friction fitted manner. The distal fastener is particularly configured to keep the seal attached to the barrel and/or to fix the seal to the elongated container barrel. With some examples the seal is located between a distal end face of the barrel and the distal fastener of the retainer.

With other examples the seal is arranged inside the elongated container barrel. The seal may be in radial abutment with an inside of the container barrel. Here, the distal fastener prevents a longitudinal, in particular a distally directed movement of the seal relative to the container barrel. Here, the distal fastener serves to keep the seal inside the barrel. A distal end of the seal may flush with a distal end face of the container barrel.

With other examples, wherein the seal is located outside the barrel but wherein the seal is for instance in axial engagement and/or axial abutment with a distal end face of the container barrel the distal fastener serves to press the seal in longitudinal direction against the distal end of the elongated container barrel, hence against the distal end face of the elongated container barrel.

Typically and generally, the distal fastener and the proximal fastener are mutually connected in a tension stable manner. Hence, a force in longitudinal direction applied to one of the distal fastener and the proximal fastener equally or unalterably transfers to the other one of the distal fastener and the proximal fastener. Typically, a distally directed force applied to the distal fastener equally and/or unalterably transfers to the proximal fastener. Moreover, a proximally directed force applied to the proximal fastener equally and/or unalterably transfers to the distal fastener. The retainer is tensionally stable at least with regard to the longitudinal position of the distal fastener relative to the proximal fastener. In this way the elongated container barrel and the seal can be durably fixed, e.g. clamped between the distal fastener and the proximal fastener.

In a further example the distal fastener comprises a proximally facing abutment face configured to abut in longitudinal direction with at least one of a distal end face of the container barrel and a distal face of the seal. With some examples, wherein the seal is located outside, e.g. axially or longitudinally adjacent the distal end of the elongated container barrel the proximally facing abutment face of the distal fastener is in direct abutment with the distal face of the seal. Here, a proximal face of the seal is in direct abutment with the distal end face of the container barrel. In other words, the seal, e.g. implemented as a sealing disc is axially or longitudinally squeezed between the proximally facing abutment face of the distal fastener and the distal end face of the container barrel.

With other examples, wherein the seal is located inside the container barrel the distal face of the seal may flush with the distal end face of the container barrel. Here, both the distal face of the seal and the distal end face of the container barrel may be in abutment with the proximally facing abutment face of the distal fastener. Here, a radial outside surface of the seal is in sealing engagement with an inside surface of the elongated container barrel.

With other examples, wherein the seal is longitudinally or axially sandwiched between the distal fastener and the distal end face of the container barrel the proximal face of the seal is in sealing engagement with the distal end face of the container barrel. Here, only an outer rim of the proximal face of the seal is in direct axial or longitudinal sealing abutment with the distal end face of the container barrel.

In either way a liquid tight and/or gas tight sealing of the distal end of the elongated container barrel can be obtained.

According to a further example the retainer comprises an elongated retainer barrel enclosing the container barrel. The retainer barrel may be of tubular or sleeve-like shape. The retainer barrel may comprise a single-pieced and/or unitary body of tubular shape. The retainer barrel and/or its body may comprise a rigid structure. An inside diameter or cross-section of the retainer barrel may be equal to or slightly larger than an outer diameter or cross-section of the container barrel. The retainer barrel may be sized and configured to accommodate the container barrel, e.g. in a press fitted or longitudinally squeezed manner.

According to a further example the retainer comprises an insert opening at a proximal end of the elongated retainer barrel. The insert opening is shaped and/or configured to receive the elongated container barrel therethrough in longitudinal direction. In this way, the container barrel can be inserted into the retainer by a sliding movement along the longitudinal direction.

In the course of arranging or assembling the elongated container barrel in the retainer the container barrel can be inserted with its distal end into and through the insert opening provided at the proximal end of the retainer barrel. The container barrel can then be pushed in a sliding manner in distal direction relative to the container barrel until it reaches a final assembly configuration, in which, e.g. the distal end of the elongated container barrel engages the distal fastener of the retainer and/or until the proximal end of the container barrel engages the proximal fastener of the retainer.

Reaching of this final assembly configuration may be accompanied by the proximal fastener of the retainer audibly or haptically engaging with the proximal end of the elongated container barrel. This particular applies when the proximal fastener of the retainer comprises a clip or snap feature configured to engage in a form fitted manner with the container barrel.

With a further example the retainer comprises an elongated sidewall and a front face. The front face is located at a distal end of the retainer. Typically, the front face is located at a distal end of the sidewall of the retainer. The front face protrudes inwardly from the elongated sidewall. Typically, the elongated sidewall is of tubular shape. In this case, the front face protrudes radially inwardly from the elongated sidewall. The front face may form or constitute a radially inwardly extending flange at the distal end of the sidewall of the retainer.

The front face and the elongated sidewall may be integrally formed. The retainer may be made of a plastic material, in particular of a thermoplastic material. The retainer may be manufactured by way of injection molding. Hence, the retainer may comprise a unitary or one-piece injection molded plastic component.

With some examples the front face of the retainer substantially coincides with the distal fastener. In other words, the distal fastener may be formed or constituted by the front face. Typically, the proximally facing abutment face of the distal fastener is provided by a proximally facing portion of the front face, e.g. facing inwardly into the interior of the retainer. The front face, in particular an inside surface of the front face may form the proximally facing abutment face of the distal fastener. Hence, the front face may act as a stopper to engage with at least one of the distally facing distal end face of the elongated container barrel and a distal face of the seal.

Typically, the front face forms or constitutes a front wall against which the distal end of the elongated container barrel may abut.

With a further example the front face comprises an aperture. Typically, the aperture is configured to receive an injection needle. The aperture typically comprises a through opening extending in longitudinal direction through the front face or front wall of the retainer. The aperture may be located in a radial center of the front face. The aperture is configured to receive an injection needle therethrough. The injection needle may comprise a double tipped injection needle. The injection needle may comprise a proximal needle tip and a distal needle tip. With the proximal needle tip the injection needle is configured to pierce and/or to penetrate the seal of the medicament container.

The injection needle is typically provided by a separate needle assembly, e.g. by a needle hub. The needle hub may comprise a fastener configured to detachably engage with a counter fastener of the medicament container. The counter fastener of the medicament container is typically provided on an outside of the sidewall adjacent to the front face of the retainer. The counter fastener of the retainer typically comprises an outer threaded portion. The fastener of the needle hub typically comprises an inner thread to threadedly engage with the outer thread of the retainer. In this way, a needle hub can be detachably arranged and/or fastened to the medicament container.

Compared to prior art solutions, wherein a vitreous cartridge is arranged inside a cartridge holder of an injection device the presently proposed medicament container represents a combination of a cartridge and a cartridge holder. Typically, the retainer does not only serve to fix or to attach the seal to the elongated container barrel but also provides a fastening of a needle hub to the medicament container. With some examples the medicament container may also comprise at least one further fastener configured to engage with a complementary shaped counter fastener of a body or housing of an injection device. Hence, the retainer may effectively replace a conventional cartridge holder of a pen-type injector. The retainer may provide a threefold function. First of all it serves to fix the distal seal to the elongated container barrel. Second, the retainer provides a mount for an injection needle. Third, the retainer provides a fastening of the medicament container to a housing or to a body of an injection device.

The medicament container is typically implemented as a disposable component. Once the content or the liquid medicament provided inside the medicament container has been used up the entirety of the medicament container is intended to become discarded.

According to a further example the elongated sidewall of the retainer comprises an outer threaded section adjacent to the front face. This outer threaded section is configured to mate or to threadedly engage with a counter threaded section of a needle hub. The counter threaded section typically comprises or forms an inner thread. In this way an injection needle and hence a needle hub can be detachably mounted and fixed to the distal end of the retainer and hence of the medicament container.

According to a further example the retainer comprises at least a first elongated shaft portion and a second elongated shaft portion. The first and the second elongated shaft portions constitute or confine a receptacle. The receptacle is configured to receive or to enclose the container barrel. With some examples the first and the second shaft portions form or constitute the elongated sidewall of the retainer. Hence, the elongated sidewall of the retainer may be formed by at least first and second elongated shaft portions. With some examples the retainer comprises more than just two elongated shaft portions. The retainer may comprise three, four or even more elongated shaft portions. Typically, the elongated shaft portions are of equal size and/or equal geometry. Each one of the first and second elongated shaft portions forms a portion of the cylindrical or tubular shaped sidewall of the retainer.

Typically, the at least first elongated shaft portion and the at least second elongated shaft portion are rigidly connected to each other. Effectively and e.g. apart from a mechanical deformation due to the elasticity of the material the elongated shaft portions are made of, the shaft portions are non-movable relative to each other. They might be implemented as portions or sections of the retainer, wherein the container may be of rigid structure and/or may be unitarily formed.

The shaft portions may be separated by windows or through openings extending through the sidewall of the retainer. The elongated shaft portions may each comprise a semicircular or arched shaped cross-section. The first and the second elongated shaft portions are typically symmetrically arranged with regard to the longitudinal central axis of the elongated container barrel. The first and second elongated shaft portion and the receptacle formed therebetween provide a tight and/or slip free arrangement of the elongated container barrel therebetween.

Typically, the first and the second shaft portions are integrally formed. The first and the second elongated shaft portions may be mutually interconnected near or at the distal end of the retainer. The distal end of the retainer may comprise a sleeve-like shape void of any recesses. The first and the second elongated shaft portions extend in longitudinal direction. They may be located diametrically opposite to each other with regard to the central axis of the retainer.

Typically, the first and the second elongated shaft portions are separated from each other by at least one or two longitudinal slits. With some examples there are provided two longitudinal slits that are arranged diametrically opposite to each other with regard to the central and longitudinal axis of the retainer. The slits extend in longitudinal direction and divide the sidewall of the retainer into first and second shaft portions.

According to another example at least one of the first elongated sidewall portion and the second elongated sidewall portion comprises an inwardly protruding protrusion. Typically and with some examples the inwardly protruding protrusion is located at a proximal end of the retainer. The inwardly protruding protrusion may provide or may comprise the above mentioned distally facing abutment face of the proximal fastener which is configured to abut in longitudinal direction with a proximal end face of the container barrel. By providing the distally facing abutment face on at least one of the first and second elongated sidewall portions of the retainer an effective axial or longitudinal clamping of the elongated container barrel and the seal between the distally facing abutment face of the proximal fastener and the proximally facing abutment face of the distal fastener can be provided and obtained.

With some examples both, namely the first elongated shaft portion and the second elongated shaft portion comprise an inwardly protruding protrusion and hence a distally facing abutment face for engagement with the proximal end face of the elongated container barrel. By providing both, the first elongated sidewall portion and the second elongated sidewall portion with the inwardly protruding protrusion the mechanical engagement of the proximal fastener with the proximal end of the elongated container barrel can be improved.

According to another example the inwardly protruding protrusion of at least one of the first elongated sidewall portion and the second elongated sidewall portion comprises a distally facing abutment face which is configured to abut with a proximal end face of the container barrel. This distally facing abutment face substantially coincides or is identical to the distally facing abutment face of the proximal fastener. In other words, the inwardly protruding protrusion may form, may constitute or may contribute to the proximal fastener.

Once a final assembly configuration has been reached and when the proximal end face of the elongated container barrel passes by the inwardly extending protrusions of the elongated sidewall portion or elongated sidewall portions the respective sidewall portion or sidewall portions will flex or relax inwardly so that the distally facing abutment face of the protrusion axially or longitudinally engages, i.e. abuts with the proximally facing proximal end face of the container barrel.

The final assembly configuration is typically reached when the distal end face of the container barrel gets in abutment with the distal fastener, in particular with the proximally facing abutment face of the distal fastener.

In another example the first elongated shaft portion and the second elongated shaft portion are mutually interconnected by at least two or several transverse web sections. The web section or web sections extend across the longitudinal slit extending between the first and the second elongated sidewall portions. The transverse web sections typically extend along the circumference of the tubular shaped sidewall. The at least two web sections connect the first elongated sidewall portion and the second elongated sidewall portion. The web sections and the sidewall portions may be integrally formed. They may be unitarily formed in an injection molded plastic component.

Typically, the proximal end of the first elongated sidewall portion and the second elongated sidewall portion is free of such web sections. In this way, at least the proximal end of the elongated sidewall portions are allowed to flex or to elastically deform radially outwardly during and/or for insertion of an elongated container barrel into the receptacle formed by the first and second elongated sidewall portions. Hence, the proximal end of the first and the second elongated sidewall portions is free or void of any interconnecting web sections.

In general, the at least two web sections increase the stability and robustness of the medicament container.

Between longitudinally adjacently arranged web sections there are provided recesses or windows in the elongated sidewall of the retainer. Such windows, through openings or recesses allow visual inspection of the content of the elongated container barrel.

Typically, the at least two or several transverse web sections are located at a predefined distance from the proximal end of the retainer. In this way it is guaranteed, that the proximal ends of the first elongated sidewall portion and the second elongated sidewall portion may flex or elastically deform in radial direction so as to enable a snap-fit engagement between the proximal fastener and the proximal end of the elongated container barrel.

Typically, the elongated container barrel is transparent. The elongated container barrel may be color dyed. The elongated container barrel may be made of a vitreous material. The elongated container barrel may comprise a glass barrel. With some other examples the elongated container barrel is made of a plastic material, in particular of a thermoplastic and transparent or color dyed plastic material. The container barrel may be substantially inelastic and may hence comprise a rigid structure.

According to another example the retainer is made of a flexible plastic material. Moreover, the retainer is made of an injection molded thermoplastic material. The retainer is a one-piece or unitarily formed plastic component. In this way, the retainer can be produced and manufactured in a rather cost efficient way thus enabling a rather straight forward cost efficient mass manufacturing of the medicament container.

According to another example at least one of a proximal end of the first elongated sidewall portion and a proximal end of the second elongated sidewall portion is flexibly deformable in a transverse direction or radial direction relative to the other one of the proximal end of the first sidewall and the proximal end of the second sidewall portion.

With some examples, the first and second elongated sidewall portions are flexibly deformable relative to each other, in particular near or at their proximal end.

In either way, the inner diameter of the retainer and hence of the elongated sidewall can be slightly widened or enlarged in the course of inserting the elongated container barrel into the retainer or into the receptacle formed or confined by the first and second elongated sidewall portions. By having at least one longitudinally extending slit at or near the proximal end of the sidewall, wherein the slit separates the first and the second elongated sidewall portions, a flexible, resilient and/or elastic temporal radial widening of the proximal end of the retainer can be provided thus enabling a sliding insertion of the elongated container barrel in distal direction into the retainer.

At least the proximal ends of the first and the second elongated sidewall portions of the retainer are separated from each other by a slit extending in longitudinal direction. In this way, the proximal portions of the sidewall portions and/or the respective protrusions provided at the proximal end of the sidewall portions can be urged radially outwardly in the course of insertion of the elongated container barrel.

According to a further example a longitudinal distance between the proximal fastener and the distal fastener is less than or equal to the longitudinal dimension or longitudinal extension of the container barrel. Hence, the longitudinal or axial distance between the proximally facing abutment face of the distal fastener and the distally facing abutment face of the proximal fastener is less than or equal to the longitudinal distance between the distal end face of the container barrel and the proximal end face of the container barrel.

In this way, the container barrel can be axially or longitudinally clamped between the distal fastener and the proximal fastener in a slack free or slip free way. One advantage would be for the longitudinal distance between the proximal fastener and the distal fastener to be slightly less than the longitudinal dimension or longitudinal extension of the container barrel. The snap fit connection between proximal and distal fasteners and proximal and distal end faces of the container barrel is accompanied by a slight but non-neglecting pretension of the retainer. In this way a rather slip free or slack free and hence a rather tight fastening of the elongated container barrel inside the retainer can be obtained.

With some other examples the longitudinal distance between the proximal fastener and the distal fastener is less than or equal to the sum of the longitudinal dimension of the container barrel and the longitudinal thickness of the seal. The seal can be sandwiched or clamped in longitudinal direction between the distal end face of the container barrel and the distal fastener of the retainer. The distance between the distally facing abutment face of the proximal fastener and the proximally facing abutment face of the distal fastener is less than or equal to the longitudinal thickness of the seal and the longitudinal dimension or longitudinal extension of the elongated container barrel.

Typically, the distance between the proximal fastener and the distal fastener is slightly less than the sum of the longitudinal dimension of the container barrel and the thickness of the seal in longitudinal direction. In this way and when reaching a final assembly configuration it is provided that the seal, typically made of an elastomeric material, is at least slightly squeezed or compressed in longitudinal direction between the distal fastener and the distal end face of the elongated container barrel. In this way a rather tight and slack free arrangement of the elongated container barrel inside the retainer can be obtained. Moreover, the seal can be compressed to a predefined degree thereby increasing its sealing capability.

The press fitting, form fitting or the positive fit of the elongated container barrel and the seal inside the retainer is also beneficial to increase the robustness of the medicament container. The retainer serving as an outer shell or serving as an outer barrel for the elongated container barrel provides a mechanical shock absorption to a predefined degree. The elongated container barrel, typically made of a vitreous and hence brittle material will become less prone to damaging or cracking since the entire outer surface of the elongated container barrel will be effectively inaccessible from outside since the container barrel is entirely enclosed inside the retainer made of a plastic material. The retainer may provide a protective shell for the elongated container barrel and may increase the mechanical resistance or robustness of the medicament container against mechanical load or mechanical shock.

In a further example the container barrel is sealed in proximal direction by a piston slidably arranged inside the container barrel. An interior volume of the medicament container confined by the piston, the seal and an inside of the container barrel is filled with the medicament, typically with the liquid medicament. The piston and the seal may be made of the same or similar materials. The seal and/or the piston can be made of an elastomeric material, such as natural or synthetic rubber. The piston and/or the seal can comprise bromobutyl rubber and/or chlorobutyl rubber. These materials exhibit good sealing capabilities with regard to the elongated container barrel.

Typically, the piston is located at or near the proximal end of the elongated container barrel. Only a distal surface of the piston is in contact with the medicament. A proximal surface of the piston serves as a thrust receiving surface which is configured to be pushed in distal direction by a drive mechanism, e.g. by a piston rod of an injection device.

According to another aspect the disclosure further relates to a drug delivery device comprising a housing and a drive mechanism. The drive mechanism comprises a piston rod configured to urge against a piston of a medicament container. The drug delivery device, e.g. implemented as an injection device, such as a handheld pen-type injector further comprises a medicament container as described above. The medicament container is assembled inside a housing of the drug delivery device or the medicament container may constitute or contribute to the housing of the drug delivery device.

With one example the medicament container as described above comprises at least one fastener at or near a proximal end of the retainer. This fastener is configured to releasably or non-releasably engage with a counter fastener provided on a body of the drug delivery device. The body of the drug delivery device may form or constitute a proximal housing component of the drug delivery device. The retainer of the medicament container may directly engage in a releasable or non-releasable manner with the body of the injection device. A distal end of the medicament container may then provide a mount for an injection needle, which when appropriately mounted to the distal end of the medicament container penetrates the seal of the medicament container to gain access to the interior of the medicament container.

In a further aspect the disclosure also relates to a method of assembly of the medicament container as described above. The method comprises the step of inserting a piston in a proximal end of an elongated container barrel, filling the interior volume confined by the piston and an inside of the sidewall of the container barrel with a liquid medicament, arranging a seal at the distal end of the elongated container barrel such that the seal extends across the distal container cross-section and inserting the preassembly of elongated container barrel, piston, medicament and seal into a retainer to fix the seal to the distal end of the elongated container barrel.

With some examples of the method it is intended, that the preassembly consisting of the elongated container barrel, the piston, the medicament and the seal is kept stationary whereas the retainer is moved relative to the elongated container barrel until a final assembly configuration has been reached, in which the proximal fastener of the retainer engages with the proximal end, e.g. with the proximal end face of the container barrel.

The proximal fastener and the proximal end of the container barrel may engage by way of a snap fit connection, which is straight forward and intuitive for both, manual and automated assembly, e.g. in a mass manufacturing environment.

It should be noted that any features, effects and benefits described above in connection with the medicament container and the drug delivery device equally apply to the method of assembly of the medicament container; and vice versa.

According to another aspect the present disclosure also relates to a method of manufacturing a medicament container for accommodating a liquid medicament. The method is particularly adapted to provide a medicament container as described above. In first method steps an elongated container barrel and an elongated retainer are provided. The elongated container barrel defines a longitudinal direction. The elongated container barrel comprises a proximal end and a distal end. The proximal end is located opposite the distal end.

Typically, the container barrel comprises a proximal container cross-section at the proximal end and the elongated container barrel comprises a distal container cross-section at the distal end. The proximal container cross-section is substantially identical to the distal container cross-section. In other words, the elongated container barrel is of substantially tubular shape.

The elongated retainer is sized and configured to enclose the elongated retainer barrel. The elongated retainer comprises a proximal fastener, typically at a proximal end of the elongated retainer and a distal fastener, typically at a distal end of the elongated retainer. The elongated retainer may comprise an elongated retainer barrel sized and shaped to accommodate or to receive the elongated container barrel entirely therein.

Once the container barrel and the retainer have been provided a seal is either arranged at the distal end of the elongated container barrel, such that the seal extends across a distal container cross-section, or the seal is arranged inside the elongated retainer.

Thereafter, the elongated container barrel is inserted in longitudinal direction into the elongated retainer and the elongated container barrel is engaged with at least one of the proximal fastener and the distal fastener of the elongated retainer. Inserting of the elongated container barrel into the elongated retainer along the longitudinal direction allows and enables to make use of a unitarily shaped or single pieced elongated retainer. The design of the elongated retainer may be kept rather simple and cost efficient. Moreover, a longitudinally directed insertion of the elongated container barrel into the elongated retainer is of practical use for a mass manufacturing process.

Typically, the present method is implementable with those components of a medicament container, namely with an elongated container barrel, a seal and a retainer as described above. Insofar all features, effects and benefits obtainable with the medicament container as described above equally apply to the method of manufacturing a medicament container; and vice versa.

Inserting of the elongated container barrel in longitudinal direction into the elongated retainer may be stopped by the distal fastener of the retainer. The distal fastener of the retainer may engage with at least one of the distal end of the elongated container barrel and the seal.

With some examples the seal is arranged inside the elongated retainer separately from the elongated container barrel. With other examples the seal is pre-assembled to or into the elongated container barrel before the preassembly of the elongated container barrel and is inserted into the elongated retainer.

With some examples, the seal may be arranged at or near the distal fastener of the retainer separately, i.e. before the elongated container barrel is inserted into the elongated retainer. With other examples, the seal is arranged at an arbitrary longitudinal position inside the retainer. In the course of inserting the container barrel in longitudinal direction into the elongated retainer the seal may be pushed in distal direction relative to the elongated container barrel until it arrives in a final assembly position or final assembly configuration.

In a final assembly position or final assembly configuration the seal may be axially clamped or squeezed between a distal end of the elongated container barrel and a proximally facing abutment face of the distal fastener of the elongated retainer.

According to a further example the retainer comprises an elongated retainer barrel configured to enclose the container barrel. The retainer comprises an insert opening at a proximal end of the elongated retainer barrel. Inserting of the elongated container barrel into the elongated retainer comprises sliding of the elongated container barrel in longitudinal distal direction through the insert opening. The seal may be either preassembled inside the elongated retainer or the seal may be arranged at the distal end of the elongated container barrel.

When arriving in a final position of assembly, hence when the elongated container barrel has been inserted completely into the hollow elongated retainer at least one of the proximal fastener and the distal fastener engages with the elongated container barrel and fixes the elongated container barrel relative to the elongated retainer. Typically, when arriving in the final position of assembly, both, the proximal fastener and the distal fastener engage with the elongated container barrel and/or with the seal.

Typically, the distally directed movement of the elongated container barrel relative to the elongated retainer is stopped or delimited by the distal fastener, e.g. providing a proximally facing abutment face for the container barrel and/or the seal.

According to another example engaging of the elongated container barrel with at least one of the proximal fastener and the distal fastener comprises establishing one of a snap joint or clip joint between the proximal fastener at the proximal end of the elongated container barrel. Typically and after completely inserting the container barrel in longitudinal direction into the elongated retainer and when arriving in a final position of assembly the proximal end of the elongated container barrel has passed beyond the proximal fastener.

The proximal fastener may provide at least one of a snap joint or clip joint. Passing the proximal fastener in distal direction allows and supports engagement of the at least one snap joint or clip joint with the proximal end of the elongated container barrel. At the same time a distal end of the elongated container barrel may be in axial or longitudinal abutment with the proximally facing abutment face of the distal fastener. In this way the elongated container barrel can be longitudinally fixed inside the elongated retainer, typically inside the elongated retainer barrel. It may be compressed in longitudinal direction between the proximal fastener and the distal fastener of the elongated retainer.

Providing of at least one of a snap joint or clip joint at the proximal fastener is of particular benefit to obtain a well-defined mechanical fixing between the elongated retainer and the elongated container barrel. The snap joint or clip joint is elastically deformable with regard to the radial direction at least during longitudinal insertion of the elongated container barrel into the elongated retainer. When a final assembly position of the container barrel has been reached inside the retainer barrel the snap joint or clip joint is free to move radially inwardly so as to engage with a proximally facing end of the container barrel.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:
H-(Lys)4-des Pro36, des Pro37 Exendin-4(1-39)-NH2,
H-(Lys)5-des Pro36, des Pro37 Exendin-4(1-39)-NH2,
des Pro36 Exendin-4(1-39),
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39); or
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39),
   wherein the group -Lys6-NH2 may be bound to the C-terminus of the Exendin-4 derivative;
   or an Exendin-4 derivative of the sequence
des Pro36 Exendin-4(1-39)-Lys6-NH2 (AVE0010),
H-(Lys)6-des Pro36 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro36 [Trp(O2)25, Asp28] Exendin-4(1-39)-Lys6-NH2,
H-des Asp28 Pro36, Pro37, Pro38 [Trp(O2)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-NH2,
H-(Lys)6-des Pro36 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(O)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-NH2,
H-Lys6-des Pro36 [Met(O)14, Trp(O2)25, Asp28] Exendin-4(1-39)-Lys6-NH2,
H-des Asp28 Pro36, Pro37, Pro38 [Met(O)14, Trp(O2)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(O2)25, Asp28] Exendin-4(1-39)-NH2,
des Pro36, Pro37, Pro38 [Met(O)14, Trp(O2)25, Asp28] Exendin-4(1-39)-(Lys)6-NH2,
H-(Lys)6-des Pro36, Pro37, Pro38 [Met(O)14, Trp(O2)25, Asp28] Exendin-4(S1-39)-(Lys)6-NH2,
H-Asn-(Glu)5-des Pro36, Pro37, Pro38 [Met(O)14, Trp(O2)25, Asp28] Exendin-4(1-39)-(Lys)6-NH2;
   or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE FIGURES

In the following, numerous examples of the injection device comprising a filling level indicator will be described in greater detail by making reference to the drawings, in which:

FIG. 1 shows a schematic illustration of a drug delivery device equipped with a medicament container,

FIG. 2 is another schematic exploded view of the injection device according to claim 1,

FIG. 3 illustrates a prior art solution of a cartridge containing a liquid medicament,

FIG. 4 shows a cross-section through a medicament container according to the present disclosure,

FIG. 5 is a perspective illustration of the medicament container,

FIG. 6 is an exploded view of the numerous components of the medicament container,

FIG. 7 is a side view of the retainer of the medicament container,

FIG. 8 is a rear view of the medicament container according to FIG. 5,

FIG. 9 is a front view of the medicament container according to FIG. 5,

FIG. 10 is a longitudinal cross-section through the retainer as illustrated in FIG. 7, and

FIG. 11 is a longitudinal cross-section through the elongated container barrel.

### DETAILED DESCRIPTION

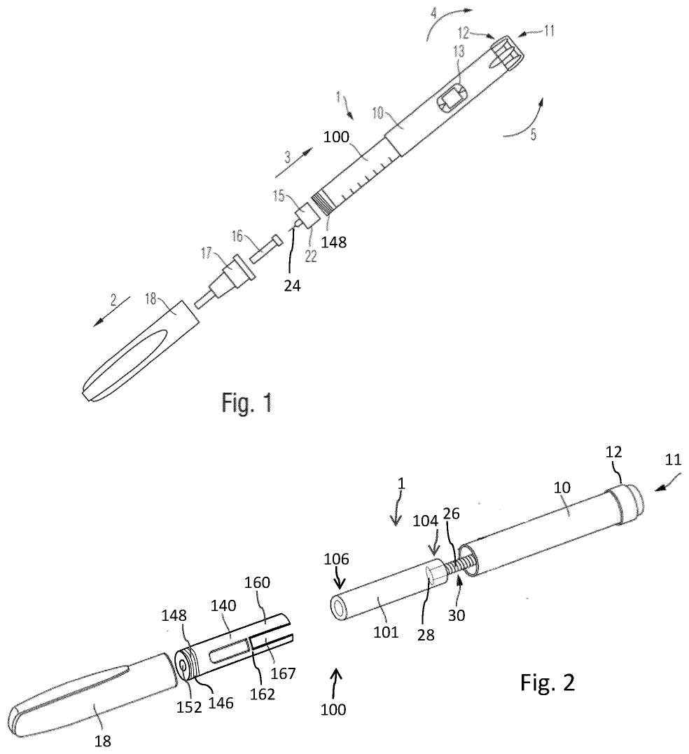

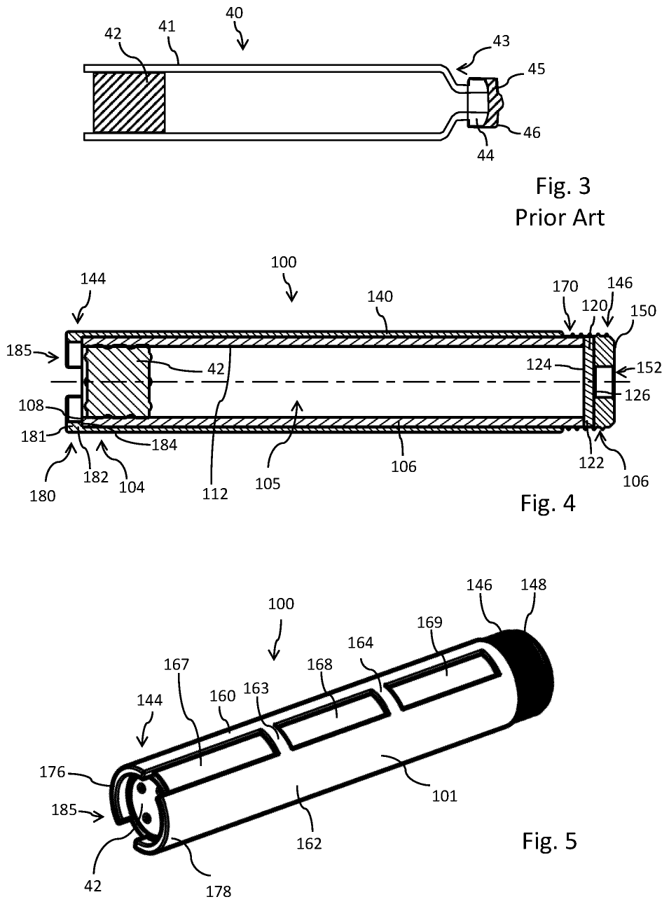

The injection device 1 as shown in FIGS. 1 and 2 can be implemented as a pre-filled disposable injection device that comprises a housing 10 to which a needle hub or needle assembly 15 can be affixed. An injection needle 24 of the needle assembly 15 is protected by an inner needle cap 16 and either an outer needle cap 17 or a protective cap 18 that is configured to enclose and to protect a distal section of the housing 10 of the injection device 1. The housing 10 may comprise and form a main housing part configured to accommodate a drive mechanism 30. The injection device 1 further comprises a container 100 the details of which will be described below. The container 100 comprises a drug container or medicament container. It may be permanently or releasably connected to the main housing 10. The container 100 is filled with a liquid medicament.

The needle assembly 15 comprises a threaded needle hub 22 configured for a screwed connection with a threaded section 148 at a distal end 146 of the container 100. As illustrated in FIG. 2, the very distal end of the container 100 comprises an aperture 152 a through opening configured to receive a proximally extending tipped end of the injection needle 24. In particular, the injection needle 24 of the needle assembly 15 intersects the needle hub 22 and comprises a double-tipped cannula.

The drive mechanism 30 comprises a piston rod 26 operable to urge a piston 42 of the container 100 in distal direction 2 in order to expel the medicament from the medicament container 100. A distal end of the piston rod 26 typically comprises a pressure piece 28 having a radially widened structure compared to the elongated piston rod 26. The pressure piece 28 will get in direct mechanical contact with a proximal face of the piston 42. The piston rod 26 may comprise a threaded rod that is in threaded engagement with the housing 10.

The drug delivery device 1 comprises a dose dial 12 and a trigger 11. In the example as illustrated in FIG. 1 the dose dial 12 and the trigger 11 are provided at a proximal end of the housing 10. The injection device 1 further comprises a dosage window 13 in which a size of a dose of the medicament currently set is visually indicated. For setting of a dose the user simply rotates the dose dial 12 in a dose incrementing direction 4 relative to the housing 10. During setting of a dose consecutive increasing numbers will show up in the dosage window 13. If a dose currently set would be too high the user has the possibility to decrease the size of a dose by dialing or rotating the dose dial 12 in the opposite, hence dose decrementing direction 5. Once a dose of appropriate size has been set or selected a dose dispensing procedure may be triggered or controlled by depressing of the trigger 11, e.g. forming a proximal end face of the injection device. The trigger 11 may be depressed in distal direction 2 by a thumb of a user in order to initiate and/or to control a dispensing operation of the injection device 1.

The medicament container 100 is illustrated in greater detail in FIGS. 2 and 4-11. The medicament container 100 comprises an elongated container barrel 101. The container barrel 101 may comprise a sleeve made of a vitreous or plastic material. The elongated container barrel 101 comprises a tubular sidewall 102. The sidewall 102 comprises a constant diameter or constant cross-section in longitudinal direction, hence in longitudinal distal direction 2 and in longitudinal proximal direction 3. The elongated container barrel 101 comprises a proximal longitudinal end 104 and an oppositely located longitudinal distal end 106. As illustrated in FIG. 11 the proximal end 104 comprises a proximal container cross-section 114. Likewise the distal end 106 comprises a distal container cross-section 116. Since the container barrel 101 is of rather straight and tubular shape and since the elongated container barrel 101 is void of a neck portion, void of a head portion and void of a shoulder portion the proximal container cross-section 114 is substantially identical to the distal container cross-section 116.

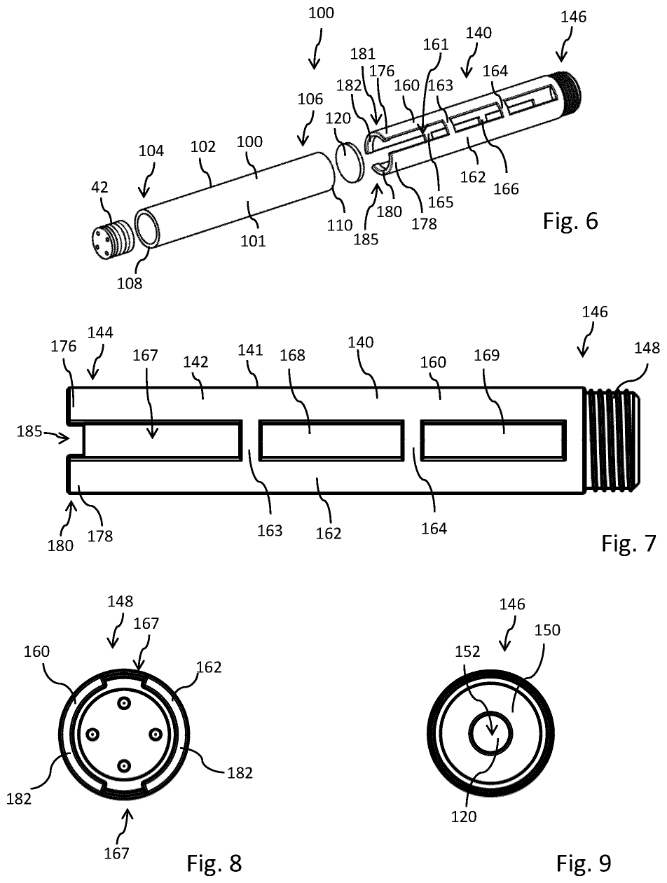

The medicament container 100 further comprises a seal 120 as shown in FIG. 6 and FIG. 4. The seal 120 may comprise a sealing disc 122. The sealing disc 122 comprises a proximal face 124 and an oppositely located distal face 126. As indicated in FIG. 4, the seal 120 is configured to seal the distal end 106 of the elongated container barrel 101. For this, an outer section of the proximal face 124 is in abutment with a distal end face 110 of the sidewall 102 of the container barrel 101.

The seal 120 is fixed and arranged at the distal end 106 with the help of a retainer 140. The retainer 140 comprises or constitutes a retainer barrel 141. The retainer 140 or the retainer barrel 141 comprises a tubular or cylindrically shaped sidewall 142. The retainer 140 comprises a proximal end 144 provided with a proximal fastener 180. The retainer 140 further comprises a distal fastener 170. The distal fastener 170 is configured to engage with the seal 120 and to keep the seal 120 in abutment with the distal end 106 of the container barrel 101. The proximal fastener 180, rigidly connected in a tension stable way with the distal fastener 170 is configured to engage, e.g. to but axially or longitudinally with the proximal end face 108 of the container barrel 101.

For this the distal fastener 170 comprises a proximally facing abutment face 174 configured to abut in longitudinal direction with the distal face 126 of the seal 120. Likewise, the proximal fastener 180 comprises a distally facing abutment face 184 to engage or to abut with the proximal end face 108 of the elongated container barrel 101.

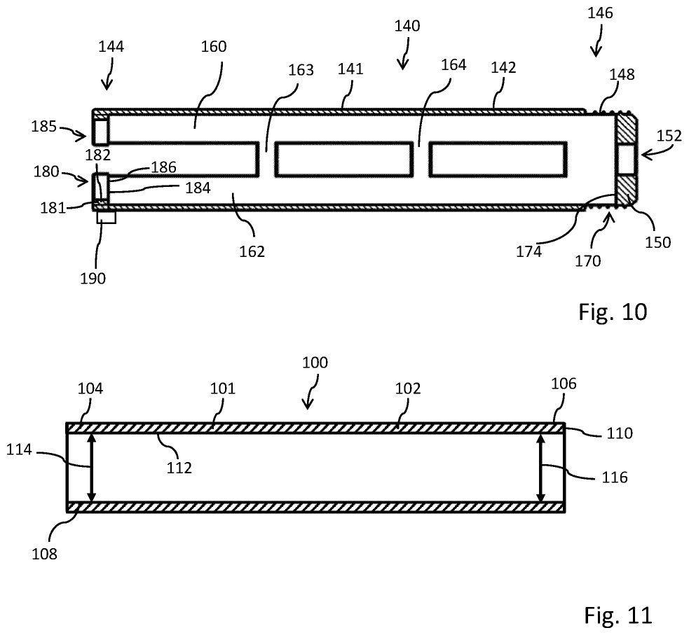

As becomes apparent from FIGS. 4 and 10 the proximal fastener 180 comprises a radially inwardly extending protrusion 181 protruding inwardly from the sidewall 142 of the retainer 140. The proximal fastener 180 and hence the protrusions 181 are provided and arranged at a proximal end 144 of the retainer 140. The radially inwardly extending protrusion 181 may comprise a rim 182 extending along the circular circumference of the sidewall 142. The protrusion 181 comprises a distally facing abutment face 184 configured to engage, i.e. to abut axially or longitudinally with the proximal end face 108 of the sidewall 102 of the container barrel 101.

At the proximal end 144 the retainer 140 comprises an insert opening 185 shaped and/or configured to receive the elongated container barrel 101 therethrough in longitudinal direction. Typically, the insert opening 185 comprises a diameter or cross-section that is slightly smaller than or equal to an outside diameter or cross-section of the elongated container barrel 101. In this way, the elongated container barrel 101 can be inserted with its distal end 106 into and through the insert opening 185 in longitudinal direction, typically along the distal direction 2. When the inside diameter or cross-section of the insert opening 185 is slightly smaller than the outside diameter or cross-section of the elongated container barrel 101, the insertion of the container barrel 101 into the retainer 140 will be accompanied by a temporary elastic deformation of the sidewall 142 and/or of the insert opening 185 of the retainer 140.

Moreover, the proximal fastener 180, e.g. the protrusions 181 of the proximal fastener 180 may form or constitute a snap joint with the proximal end 104 of the elongated container barrel 101. For instance, the protrusion or protrusions 181 may comprise a respective snap feature 186 configured to audibly and/or haptically engage with the proximal end 104 of the elongated container barrel 101. The snap feature 186 and the proximal end face 108 of the elongated container barrel 101 may constitute or form a snap joint.

The distal fastener 170 may be formed by a front face 150 integrally formed with the sidewall 142 of the retainer 140. The front face 150 may be provided at a distal end 146 of the retainer 140. The front face 150 may comprise or may form a radially inwardly extending flange section having a central aperture 152 extending therethrough in longitudinal direction. The aperture 152 serves to receive a proximal tip of the injection needle 24 when the needle hub 22 is attached to the distal end 146 of the retainer 140. For attachment of the needle hub 22 the retainer 140 comprises a threaded section 148 at or near the distal end 146 of the sidewall 142. In this way and since the needle hub 22 comprises a complementary shaped inner thread a threaded connection between the needle hub 22 and the retainer 140 can be provided.

An inside surface and hence a proximally facing surface 174 of the front face 150 forms or constitutes, e.g. coincides with the proximally facing abutment face of the distal fastener 170. As illustrated in FIG. 4, the inside surface of the front face 150 and hence the proximally facing abutment face 174 provides an end stop for the seal 120. In the final assembly configuration as illustrated in FIG. 4, the seal 120 is axially or longitudinally squeezed between the distal end face 110 of the container barrel 101 and the proximally facing abutment face 174 of the distal fastener 170, i.e. the proximally facing abutment face 174 of the front face 150.

As illustrated in FIG. 4, the longitudinal distance between the proximal fastener 180 and the distal fastener 170 is less than or equal to the sum of the total axial or longitudinal elongation of the elongated container barrel 101 plus the thickness of the seal 120. In this way it can be provided, that when reaching the final assembly configuration, in which the proximal fastener 180 snaps or positively engages with the proximal end face 108 of the elongated container barrel 101, the seal 120 is squeezed in longitudinal or axial direction to a predefined degree thereby providing an increased sealing capability compared to an unbiased or non-squeezed state.

An interior 105 of the elongated container barrel is sealed in distal direction by the sealing disc 120. It is sealed in proximal direction 3 by a piston 42 slidably arranged inside the tubular shaped elongated container barrel 101. The piston 42 is displaceable in distal direction 2 under the effect of the distally advancing piston rod 26.

As it becomes particularly apparent from FIGS. 6, 7, 8 and 10 the elongated sidewall 142 of the retainer barrel 141 and hence of the retainer 140 comprises a first elongated sidewall portion 160 and a second elongated sidewall portion 162. These sidewall portions 160, 162 may adopt the shape of a half cylinder. The first and second elongated sidewall portions 160, 162 are mutually interconnected by web sections 163, 164, 165, 166 as illustrated in FIG. 7. The elongated and parallel extending sidewall portions 160, 162 are mutually connected on each circumferential side edge by two web sections 163, 164 and two further web sections 156, 166 as illustrated in FIG. 6.

The web sections 164, 166 may be regarded as distal web sections because they are located distally from the two further web sections 163, 165. The further web sections 163, 165 provide or serve as proximal web sections 163, 165. The proximal web sections 163, 165 are located at a predefined distance from the proximal end 144 of the retainer 140. In this way it is provided that a proximal end 176 of the first longitudinal sidewall portion 160 is able to flex in radial direction relative to the proximal end 178 of the second longitudinal sidewall section 162. Longitudinally between the numerous web sections 163, 164 there are provided windows 167, 168, 169. The proximal window 167 is open towards the proximal end 144. It is confined in distal direction by the proximal web section 163. The middle window 168 is confined in longitudinal direction by the proximal web section 163 and the distal web section 164. The distal window 169 is confined in longitudinal direction by the threaded section 148 or the distal end 146 of the retainer 140 at a proximal direction by the distal web section 164.

The numerous windows 167, 168, 169 provide and enable visual inspection of the content of the container barrel 101. The web sections 163, 164, 165 and 166 provide structural strengthening of the longitudinal sidewall portions 160, 162. The longitudinal sidewall portions 160, 162 form or constitute a receptacle 161 therebetween. The receptacle 161 as illustrated in FIG. 6 is sized to receive or to enclose the container barrel 101. The elongated container barrel 101 can be inserted into the receptacle 161 by insertion through the insert opening 185 provided at the proximal end 144 of the retainer 140. The proximal end of the longitudinal sidewall portions 160, 162 define or delimit the insert opening 185 as seen in radial direction.

Since the proximal ends of the longitudinal sidewall portions 160, 162 may exhibit a certain degree of elasticity as seen in radial direction the diameter or cross-section of the insert opening 185 can be at least temporarily slightly increased during the process of inserting the elongated container barrel 101 into the receptacle 161 in longitudinal direction. When reaching a final assembly configuration, in which the proximal fastener 180 engages the proximal end 104 of the container barrel 101 the proximal end of the sidewall portions 160, 162 may return into an initial unbiased configuration. In this way, an inside diameter or cross-section of the insert opening 185 is typically slightly smaller than the respective outer diameter or cross-section of the elongated container barrel 101. When reaching the final assembly configuration, the proximal fastener 180 of the retainer 140 audibly and/or haptically engages with the proximal end 104 of the container barrel 101 thereby forming a snap joint.

Since the proximal ends 176, 178 of the elongated sidewall portions 160, 162 are void of interconnecting web sections they are allowed to flex radially outwardly as the medicament container 100 is inserted in distal direction 2 into the receptacle 161. When reaching a final assembly configuration as illustrated in FIG. 4, The proximal end face 108 of the elongated container barrel 101 passes by the radially inwardly extending protrusion 181 or the respective rim 182 of the proximal fastener 180 thereby locking the elongated container barrel 101 in place inside the retainer 140.

As it is further indicated in FIG. 10 the retainer 140 may be optionally provided with a further fastener 190 configured to engage with a correspondingly shaped counter fastener of the body 10 of the injection device 1. In this way the medicament container 1 can be detachably or undetachably fastened to the body 10 and may thus contribute to the housing of an injection device 1.

The interior volume 105 of the medicament container as it is confined by the distal face of the piston 42, the proximal face 124 of the seal 120 and the inside surface 112 of the sidewall 102 may be entirely filled with the liquid medicament. It may be filled void of gas cavities. Since the outlet of the medicament container 100 is sealed across the entire distal cross-section 116 by the seal 120 a rather complete and residualless emptying of the medicament container 1 can be provided as the piston 42 is urged step-by-step or continuously in distal direction 2.

In the empty configuration the distal face of the piston 42 may directly engage the proximal face 124 of the seal 120. The residual amount of medicament located between the seal 120 and the piston 42 can be thus reduced to a minimum.

### LIST OF REFERENCE NUMBERS

1 injection device
2 distal direction
3 proximal direction
4 dose incrementing direction
5 dose decrementing direction
10 body
11 trigger
12 dose dial
13 dosage window
14 cartridge holder
15 needle assembly
16 inner needle cap
17 outer needle cap
18 protective cap
20 threaded portion
22 needle hub
24 injection needle
26 piston rod
28 pressure piece
30 drive mechanism
40 cartridge
41 barrel
42 piston
43 neck portion
44 head portion
45 seal
46 cap
100 medicament container
101 container barrel
102 side wall
104 proximal end
105 interior volume
106 distal end
108 end face
110 end face
112 inside surface
114 cross section
116 cross section
120 seal
122 sealing disc
124 proximal face
126 distal face
140 retainer
141 retainer barrel
142 side wall
144 proximal end
146 distal end
148 threaded section
150 front face
152 aperture
160 shaft portion
161 receptacle
162 shaft portion
163 web section
164 web section
165 web section
166 web section
167 window
168 window
169 window
170 fastener
174 abutment face
176 proximal end
178 proximal end
180 fastener
181 protrusion
182 rim
184 abutment face
185 insert opening
186 snap feature
190 fastener

1.-19. (canceled)

20. A medicament container for accommodating a liquid medicament, the medicament container comprising:
an elongated container barrel defining a longitudinal direction and comprising a proximal end and a distal end, wherein the proximal end is opposite the distal end, wherein the elongated container barrel comprises a proximal container cross section at the proximal end, wherein the elongated container barrel comprises a distal container cross section at the distal end and wherein the proximal container cross section is substantially identical to the distal container cross section;
a seal arranged at the distal end and extending across the distal container cross section; and
a retainer engaged with the seal and engaged with the elongated container barrel to fix the seal to the distal end.

21. The medicament container of claim 20, wherein the retainer comprises a proximal fastener engaged with the proximal end of the elongated container barrel.

22. The medicament container of claim 21, wherein the proximal fastener comprises a distally facing abutment face configured to abut in longitudinal direction with a proximal end face of the elongated container barrel.

23. The medicament container of claim 20, wherein the retainer comprises a distal fastener engaged with at least one of the distal end of the elongated container barrel and the seal.

24. The medicament container of claim 23, wherein the distal fastener comprises a proximally facing abutment face configured to abut in a longitudinal direction with at least one of a distal end face of the elongated container barrel and a distal face of the seal.

25. The medicament container of claim 20, wherein the retainer comprises an elongated retainer barrel enclosing the elongated container barrel.

26. The medicament container of claim 25, wherein the retainer comprises an insert opening at a proximal end of the elongated retainer barrel, wherein the insert opening is shaped or configured to receive the elongated container barrel therethrough in longitudinal direction.

27. The medicament container of claim 20, wherein the retainer comprises an elongated sidewall and a front face, wherein the front face is located at a distal end of the retainer and wherein the front face protrudes inwardly from the elongated sidewall.

28. The medicament container of claim 27, wherein the front face comprises an aperture configured to receive an injection needle.

29. The medicament container of claim 27, wherein the elongated sidewall comprises an outer threaded section adjacent to the front face.

30. The medicament container of claim 20, wherein the retainer comprises at least a first elongated shaft portion and a second elongated shaft portion, the first and the second elongated shaft portions constitute or confine a receptacle configured to receive or to enclose the elongated container barrel.

31. The medicament container of claim 30, wherein at least one of the first elongated shaft portion and the second elongated shaft portion comprises an inwardly protruding protrusion at a proximal end of the retainer.

32. The medicament container of claim 30, wherein the first elongated shaft portion and the second elongated shaft portion are mutually interconnected by at least two or several transverse web sections.

33. The medicament container of claim 30, wherein at least one of a proximal end of a first shaft portion and a proximal end of a second shaft portion is flexibly deformable in transverse direction relative to the other one of the proximal end of the first shaft portion and the proximal end of the second shaft portion.

34. The medicament container of claim 22, wherein the retainer comprises a distal fastener engaged with at least one of the distal end of the elongated container barrel and the seal, and wherein a longitudinal distance between the proximal fastener and the distal fastener is less than or equal to a sum of a longitudinal dimension of the elongated container barrel and a thickness of the seal in longitudinal direction.

35. The medicament container of claim 20, wherein the elongated container barrel is sealed in proximal direction by a piston slidably arranged inside the elongated container barrel and wherein an interior volume confined by the piston, the seal and an inside of the elongated container barrel is filled with the medicament.

36. A method of manufacturing a medicament container for accommodating a liquid medicament, the method comprising:
providing an elongated container barrel defining a longitudinal direction and comprising a proximal end and a distal end, wherein the proximal end is opposite the distal end;
providing an elongated retainer configured to enclose the elongated container barrel, the elongated retainer comprises a proximal fastener and a distal fastener;
arranging of a seal at the distal end of the elongated container barrel such that the seal extends across a distal container cross-section, or arranging of the seal inside the elongated retainer;
inserting of the elongated container barrel in longitudinal direction into the elongated retainer; and engaging of the elongated container barrel with at least one of the proximal fastener and the distal fastener.

37. The method of claim 36, wherein the retainer comprises an elongated retainer barrel configured to enclose the elongated container barrel and wherein the elongated retainer comprises an insert opening at a proximal end of the elongated retainer barrel.

38. The method of claim 37, wherein inserting of the elongated container barrel into the elongated retainer comprises sliding of the elongated container barrel in longitudinal distal direction through the insert opening.

39. The method of claim 36, wherein engaging of the elongated container barrel with at least one of the proximal fastener and the distal fastener comprises establishing one of a snap joint or clip joint between the proximal fastener and the proximal end of the elongated container barrel.
